(19) United States
(12) Patent Application Publication
(10) Pub. No.: US 2023/0383256 A1
(43) Pub. Date: Nov. 30, 2023

(54) PROCESS FOR PRODUCING CULTURED RED BLOOD CELLS

(71) Applicant: Erypharm, Paris (FR)

(72) Inventors: Guillaume ROUSSEAU, Paris (FR); Luc DOUAY, Paris (FR); Marie-Catherine GIARRATANA, Saint-Ouen (FR); Florent MATHIEU, Saint-Germain-en-Laye (FR)

(21) Appl. No.: 18/246,471

(22) PCT Filed: Sep. 23, 2021
(86) PCT No.: PCT/EP2021/076259

§ 371 Date: Mar. 23, 2023

(30) Foreign Application Priority Data
Sep. 23, 2020 (FR) FR2009679

(51) Int. Cl. C12N 5/078; A61K 35/18

(52) U.S. Cl. CPC C12N 5/0641; A61K 35/18; C12N 2506/11; C12N 2511/00

(57) ABSTRACT

The invention relates to a process for producing cultured red blood cells from stem cells or cells of an immortalized cell line of the erythroid lineage.

### OBJECT OF THE INVENTION

The present invention relates to a method for producing cultured red blood cells.

### TECHNICAL BACKGROUND

Transfusion of red blood cells, or erythrocytes, is commonly used for many medical and surgical applications. This procedure alone has saved millions of lives over the past 60 years. The demand for such transfusions is expected to increase in the future due to the aging population.

Red blood cell transfusion is used as a treatment for anemia. The supply depends on voluntary blood donation, but a significant amount of labor is required for collection, preparation and storage to ensure its continuous supply. In addition, red blood cell preparations from donated blood are not completely free of residual infectious risks. In such circumstances, in order to provide a stable supply of safe red blood cells, there is an increasing need to artificially manufacture red blood cells as a supplement to blood donation.

The ex vivo production of cultured red blood cells, also designated as artificially produced, has many therapeutic and scientific interests and applications. For example, blood transfusion, drug transport, “Red blood cells as medicine” and as a test carrier for drug development.

Although many attempts have been made to date to derive red blood cells in vitro from hematopoietic stem cells and/or progenitors (derived from bone marrow, umbilical cord blood or peripheral blood), embryonic stem cells, iPSCs or immortalized cell lines of the erythroid lineage, they have failed to reach the industrial stage for a variety of reasons, including cost, protocol duration, too many steps, use of feeder cells, labor intensity, low yield, or the inability to completely differentiate erythroid cells into anucleate cells.

For example, Giarratana et al (2011) “Proof of principle for transfusion of in vitro-generated red blood cells”, Blood 118:5071-5079 describe the ex vivo production of cultured red blood cells from hematopoietic stem cells isolated from peripheral blood. However, the method used is not industrializable.

There is therefore a need for a method for the industrial production of cultured red blood cells that are safe and have a functionality similar to native red blood cells, in particular characterized by good deformability and the presence of functional hemoglobin.

### SUMMARY OF THE INVENTION

The present invention thus relates to a method for producing cultured red blood cells, in particular from fresh or frozen cells, the cells being stem cells and/or progenitors or cells of an immortalized cell line of the erythroid lineage, comprising the following steps:
a) at least one batch or fed-batch bioreactor culture of the cells;
b) perfusion bioreactor culture of the cells obtained in step a);
c) Washing and particle sorting of the cells obtained in step b) to obtain a population of cultured red blood cells.

The present invention also relates to cultured red blood cells obtained, or obtainable, by the implementation of the process defined above.

The present invention also relates to a population of cultured red blood cells, which can be obtained by carrying out the above defined process, which population of cultured red blood cells has at least 1, preferably at least 2, 3, 4, 5, 6, 7, 8, 9, 10, 11, 12, 13, 14, or all of the following features:
A percentage of Hoechst+ lower than 30%;
Optionally a percentage of CD36+ cells lower than 50%;
Optionally a percentage of CD71+ cells higher than 50%;
Optionally a percentage of cells labeled with thiazole orange higher than 50%;
A MCV of from 80 fL to 180 fL, in particular of from 80 fL to 160 fL;
A MCH higher than 24 pg/cell;
A MCHC higher than 18 g/dl, in particular higher than 19 g/dl;
A p50 of from 18 to 28 mmHg;
Optionally a proportion of HbA of from 70% to 100%;
Optionally a proportion of HbF of from 0% to 30%;
Optionally a proportion of HbA2 below 8%;
A HbCO proportion of from 0% to 10%;
A MetHb proportion of from 0% to 3%;
A deformability higher than 75% of that of native red blood cells;
An ATP content of from 4 to 12 μmol/g Hb.

The present invention also relates to a pharmaceutical composition comprising cultured red blood cells as defined above as active substance, optionally in combination with at least one pharmaceutically acceptable excipient or carrier.

The present invention also relates to cultured red blood cells as defined above, or a pharmaceutical composition as defined above, for use in a method of diagnosing, of preventing or treating a disease, or a disorder, characterized by a deficiency in red blood cells or in functional hemoglobin in an individual.

The present invention also relates to a method of diagnosing, preventing or treating a disease, or a disorder, characterized by a deficiency in functional red blood cells or hemoglobin in an individual, wherein the individual is administered an effective amount of cultured red blood cells as defined above or of a pharmaceutical composition as defined above.

The present invention also relates to the use of cultured red blood cells as defined above for the preparation of a reagent for the diagnosis, or a medicament for the prevention or treatment, of a disease, or a disorder, characterized by a deficiency in red blood cell count or in functional hemoglobin in an individual.

### DESCRIPTION OF THE INVENTION

As a preliminary remark, it should be noted that the term “comprising” means “including”, “containing” or “encompassing”, i.e. when a subject-matter “comprises” an element or several elements, elements other than those mentioned can also be included in the subject-matter. Conversely, the expression “consisting of” means “constituted of”, i.e. when an object “consists of” an element or elements, the object cannot include other elements than those mentioned.

### Cells

It is possible to consider the production of red blood cells from a variety of cell sources. The method according to the invention uses stem cells, progenitors, or cells of an immortalized cell line of the erythroid lineage as the cell source.

The stem cells may be embryonic stem cells (ESCs), induced pluripotent stem cells (iPSCs), or hematopoietic stem cells and/or progenitors (HSCs/HPs). Preferably the method according to the invention uses hematopoietic stem cells (HSCs) as the cell source.

Cells of an immortalized cell line of the erythroid lineage can be immortalized at the stage of an erythroid progenitor or an erythroid precursor. In addition, hematopoietic stem cells (HSCs) can also be immortalized.

Immortalization is preferably performed conditionally. These immortalized cells can then be passaged indefinitely in vitro, cryopreserved and recovered, and, conditionally, produce fully differentiated red blood cells from a defined and well characterized source. Conditional immortalization can be achieved by any method well known to the person skilled in the art.

Embryonic stem cells (ESCs) and induced pluripotent stem cells (iPSCs) are pluripotent stem cells. These cells are both capable of differentiation into many cell types and capable of self-replication. They can maintain this pluripotency of differentiation even after undergoing proliferation by division. Embryonic stem cells refer to pluripotent stem cells derived from blastocyst stage embryos, which is the early stage of animal development. Induced pluripotent stem cells (iPSCs) are produced by introducing several types of transcription factor genes into somatic cells such as fibroblasts.

The embryonic stem cells (ESCs) according to the invention are obtained by any means not requiring the destruction of human embryos. For example, by using the technology described by Chung et al (Chung et al, Human Embryonic Stem Cell lines generated without embryo destruction, Cell Stem Cell (2008)).

According to an embodiment of the invention, said stem cells used in the process according to the invention are not human embryonic stem cells (hESC) and/or iPSCs.

The hematopoietic stem cells (HSCs) used in the method according to the invention are multipotent cells. They are capable of differentiating into all blood cell differentiation lineages and are capable of self-replicating while maintaining their multipotency.

Cells of an immortalized cell line of the erythroid lineage are cells already committed to the erythroid lineage but capable of self-replication and under external control of differentiating into erythroid lineage cells.

The hematopoietic stem cells and/or progenitors (HSCs/HPs) used in the method according to the invention can be derived from any source, including, being derived from bone marrow, umbilical cord/placental blood or peripheral blood with or without prior mobilization.

The origin of stem cells and cells of an immortalized cell line of the erythroid lineage is not particularly limited as long as it is derived from a mammal. Preferred examples include humans, dogs, cats, mice, rats, rabbits, pigs, cows, horses, sheep, goats and the like, humans being most preferred.

The cells used in the process according to the invention can produce, without limitation, universal donor red blood cells, red blood cells of a rare blood type, red blood cells for personalized medicine (e.g., autologous transfusion, possibly with genetic engineering), and red blood cells designed to include one or more proteins of interest.

In certain embodiments which may be combined with any of the foregoing embodiments, said cells used in the method according to the invention may be isolated from a patient having a rare blood group including, without limitation, Oh, CDE/CDE, CdE/CdE, CwD−/CwD−, −D−/−D−, Rhnull, Rh: −51, LW (a−b+), LW (ab−), SsU−, SsU (+), pp, Pk, Lu (a+b−), Lu (ab−), Kp (a+b−), Kp (ab−), Js (a+b−), Ko, K: −11, Fy (ab−), Jk (ab−), Di (b−), I−, Yt (a−), Sc: −1, Co (a−), Co (ab−), Do (a−), Vel−, Ge−, Lan−, Lan (+), Gy (a−), Hy−, At (a−), Jr (a−), In (b−), Tc (a−), Cr (a−), Er (a−), Ok (a−), JMH− and En (a−).

According to an embodiment of the invention, said cells can be embryonic stem cells (ESCs), preferably human (hESCs) and preferably selected from the group consisting of H1, H9, HUES-1, HUES-2, HUES-3, HUES-7, CLOT lines and pluripotent stem cells (iPSCs), preferably human (hiPSCs) Preferably, said cells are hematopoietic stem cells (HSCs), more preferably human.

In the case of cells derived from umbilical cord/placental blood or from peripheral blood, from bone marrow, or from an apheresis collection, a specific CD34+ cell selection step can be performed before step a) of the method according to the invention.

Apheresis is a technique for the removal of certain blood components by extracorporeal circulation of blood. The components that are to be collected are separated by centrifugation and extracted, while the components that are not collected are reinjected into the (blood) donor or the patient (therapeutic apheresis).

CD34+ (positive) means that the CD (differentiation cluster) 34 antigen is expressed on the cell surface. This antigen is a marker for hematopoietic stem cells and hematopoietic progenitor cells, and disappears as they differentiate. Similar cell populations also include CD133 positive cells.

In the case where the cells of origin are ESCs, iPSCs or cells of an immortalized cell line of the erythroid lineage, pre-culture steps can be added upstream of the culture step in a batch or fed-batch bioreactor to multiply the cells and optionally to commit them to the differentiation pathway of the erythroid lineage.

Whatever the cell source, a preliminary step of freezing the starting cells is often required for transport and preservation reasons. Cell freezing methods are well known in the state of the art and include a programmed temperature descent as well as the use of cryoprotectant such as lactose or dimethylsulfoxide (DMSO). When added to the medium, DMSO prevents the formation of intracellular and extracellular crystals in the cells during the freezing process.

Thus, in a particular embodiment of the invention, the method according to the invention comprises a cell thawing step, prior to step a), in case the starting cells are frozen. Methods for thawing cells are well known to the person skilled in the art.

Thawing is a step in the method that should not be neglected, especially when DMSO has been used for freezing. This compound is indeed a cryopreservative as long as the cell suspension is maintained in liquid nitrogen or in nitrogen vapor. However, it becomes cytotoxic as soon as the cell suspension is thawed. It is therefore necessary to remove DMSO very quickly by several washing steps as soon as the cells are thawed, as is well known to the person skilled in the art.

Once the cells have been thawed, said cells are cultured in a batch or fed-batch bioreactor (step a) of the method according to the invention.

In other cases, the starting cells can be fresh, i.e. the time between the collection of the cells and the culture is short enough not to require freezing, preferably this time is less than 48 hours. This situation can exist, for example, when the sampling center is located on the same site or near the production center. In this situation, the method according to the invention begins directly with step b) of culturing in a batch or fed-batch bioreactor.

### Step a)

The purpose of the batch or fed-batch bioreactor culture(s) is to commit or differentiate the starting cells, or to enhance their commitment or differentiation, into the erythroid lineage. In other words, according to the invention, step a) is preferably continued until the cultured cells are committed to the erythroid lineage. According to the invention, cells are considered to be sufficiently committed to the erythroid lineage when they exhibit one or more specific features of the erythroid lineage, such as a percentage of cells exhibiting the CD235 marker, measurable, for example, by flow cytometry, higher than 50%, or a percentage of cells with an erythroid phenotype, measurable, for example, by cytological counting after staining with the May-Grünwald Giemsa dye, higher than 50%

One or more successive, or iterative, cultures in a batch or fed-batch bioreactor can be conducted. Step a) of the process according to the invention can thus be repeated several times, preferably between 1 and 4 times.

In “batch” cultures, the medium is not renewed, so the cells only have a limited quantity of nutrients. The “fed-batch” culture corresponds to a “batch” culture with a supply in particular of nutrients and/or of culture medium.

The bioreactor design used for cell culture in step a) is not particularly limited as long as it can generally culture animal cells. Preferably, the bioreactor in step a) has a capacity of from 0.5 to 5000 L, more preferably of from 0.5 to 500 L.

Cultures according to the invention are conducted in a bioreactor with the cells suspended in a suitable culture medium under controlled or regulated conditions, namely in particular agitation, temperature, pH, and dissolved oxygen (DO). Specific examples of bioreactors, culture conditions, and propagation methods well known to the person skilled in the art may be combined in any suitable manner to promote differentiation or commitment of the starting cells to the erythroid lineage and may be adapted depending on the type of starting cells.

The person skilled in the art is able to select or prepare a suitable culture medium according to the invention. As an example of suitable culture media one may mention those described in the international publication WO2011/101468A1 and in the article Giarratana et al. (2011) “Proof of principle for transfusion of in vitro-generated red blood cells”, Blood 118:5071-5079.

The culture medium generally comprises a basal culture medium for eukaryotic cells, such as DMEM, IMDM, RPMI 1640, MEM or DMEM/F1, which are well known to the person skilled in the art and widely available commercially.

The culture medium may also include plasma, in particular in an amount of 0.5% to 6% (v/v).

In addition, various cytokines, hormones and growth factors may be included in the culture medium, as well as other compounds, in particular low molecular weight compounds, that act on the cells.

The person skilled in the art is able to adapt the culture medium by adding certain components or by modulating the quantities of certain components, in particular sodium, potassium, calcium, magnesium, phosphorus, chlorine, various amino acids, various vitamins, various antioxidants, fatty acids, sugars and analogues, fetal bovine serum, human plasma, human serum, horse serum, transferrin, lactoferrin, heparin, cholesterol, ethanolamine, sodium selenite, monothioglycerol, mercaptoethanol, bovine serum albumin, human serum albumin, sodium pyruvate, polyethylene glycol, poloxamers, surfactants, lipid droplets, antibiotics agar, collagen, methylcellulose, various cytokines, various hormones, various growth factors, various small molecules, various extracellular matrices and various cell adhesion molecules.

Examples of cytokines comprised in the culture medium comprise interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7) interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-18 (IL-18), Interleukin-21 (IL-21), Interferon-A (IFN-α), interferon-β (IFN-β), interferon-γ (IFN-γ), granulocyte colony-stimulating factor (G-CSF), monocyte colony-stimulating factor (M-CSF), granulomacrophage colony-stimulating factor (GM-CSF), stem cell factor (SCF), flk2/flt3 ligand (FL), leukemic cell inhibitory factor (LIF), oncostatin M (OM), erythropoietin (EPO), thrombopoietin (TPO) However, it is not limited to these.

The various small molecules included in the culture medium may include aryl hydrocarbon receptor antagonists such as StemRegenin1 (SR1), hematopoietic stem cell self-renewal agonists such as UM171, and the like, without limitation.

Growth factors comprised in the culture medium may comprise transforming growth factor-a (TGF-a), transforming growth factor-β (TGE-β), macrophage inflammatory protein-1a (MIP-1a), epidermal growth factor (EGF), fibroblast growth factor-1, 2, 3, 4, 5, 6, 7, 8, or 9 (FGF-1, 2, 3, 4, 5, 6, 7, 8, 9), nerve cell growth factor (NGF), vascular endothelial growth factor (VEGF), hepatocyte growth factor (HGF), leukemia inhibitory factor (LIF), nexin I protease, nexin II protease, platelet-derived growth factor (PDGF), cholinergic differentiation factor (CDF), various chemokines, Notch ligands (such as Delta1), Wnt proteins, angiopoietin-like proteins 2, 3, 5, or 7 (Angpt 2, 3, 5, 7), insulin-like growth factors (GF), insulin-like growth factor binding protein (IGFBP), pleiotrophin, and the like, without limitation.

Hormones comprised in the culture medium may comprise hormones in particular of the glucocorticoid family such as dexamethasone or hydrocortisone, of the thyroid hormone family, such as T3 and T4, of the ACTH, of the alpha-MSH or of the insulin family.

Preferably, in order to promote the commitment or differentiation of the starting cells into the erythroid lineage, the culture medium comprises at least one erythrocyte differentiation factor, in particular selected from the group consisting of: stem cell factor (SCF), interleukin-3 (IL-3), interleukin-6 (IL-6), interleukin-11 (IL-11), flk2/flt3 ligand (FL), thrombopoietin (TPO) and erythropoietin (EPO), more preferably the culture medium comprises at least one erythrocyte differentiation factor selected from the group consisting of: Stem Cell Factor (SCF), flk2/flt3 ligand (FL), interleukin-3 (IL-3), thrombopoietin (TPO) and erythropoietin (EPO), and even more preferably the culture medium comprises one, two or three erythrocyte differentiation factors selected from the group consisting of SCF, IL-3 and EPO.

The concentration of a cytokine or a growth factor in the culture medium, particularly at the time of its addition to the culture medium, may be set within a range in which differentiation of hematopoietic stem cells and/or hematopoietic progenitor cells into erythrocytes can be achieved, and generally within a range of from 0.1 ng/ml to 1000 ng/ml, preferably from 1 ng/ml to 200 ng/ml.

The concentration of a hormone in the culture medium, especially at the time of its addition to the culture medium, can be suitably set within a range in which differentiation of the hematopoietic stem cells and/or hematopoietic progenitor cells into erythrocytes can be achieved, and generally within a range of from 0.1 ng/ml to 1000 μg/ml, preferably of from 1 μg/ml to 500 μg/ml.

Particularly preferably, the culture medium used in step a) comprises a basal culture medium for eukaryotic cells, heparin, in particular in a concentration of from 0.2 to 2 U/ml, plasma, in particular in a concentration of 0.5 to 6% (v/v), transferrin, especially in a concentration of from 100 to 500 μg/ml, insulin, in particular in a concentration of from 1 to 15 μg/ml, SCF, in particular in a concentration of from 50 to 300 ng/ml, EPO, in particular in a concentration of from 1 to 5 IU/ml, IL-3, in particular in a concentration of from 1 to 10 ng/ml and a glucocorticoid, in particular in a concentration of from 0.5 to 5 μM.

Preferably, the culture in step a) is performed for a period of time sufficient to achieve a cell concentration higher than 0.1 million cells/ml. Preferably this period of time is from 1 day to 15 days, more preferably from 3 days to 10 days, and even more preferably from 6 to 8 days.

Preferably, the culture temperature in step a) is comprised between 33° C. and 40° C., more preferably between 35° C. and 39° C., and even more preferably between 36° C. and 38° C.

Preferably, the culture pH of step a) is comprised between 7 and 8, more preferably between 7.2 and 7.7.

Preferably, the culture DO of step a) is comprised between 1% and 100%, more preferably between 10% and 100%.

Preferably, a renewal or a supply of new or fresh medium is carried out during step a), in particular to avoid intoxication of the cells by catabolites or a shortage of nutrients.

### Step b)

Following the batch or fed-batch culture(s) of step a), the cells are transferred to another bioreactor, operated in perfusion (step b)). The purpose of step b) is to multiply the cultured cells and complete their differentiation to an enucleated reticulocyte stage corresponding to a young or immature red blood cell, or to a mature red blood cell stage.

Perfusion is a continuous culture method in which cells are retained in the bioreactor or returned to the bioreactor while spent culture medium is evacuated, compensated by the addition of new or fresh culture medium. The used and discharged medium therefore contains no cells. A higher cell concentration and yield of cell products can be achieved in a perfusion bioreactor, with a reduced reaction volume, compared to a batch or fed-batch bioreactor.

Step b) is conducted in a bioreactor suitable for perfusion culture. Numerous designs of bioreactors suitable for culturing the cells in step b) are known to the person skilled in the art. Preferably, the bioreactor of step b) has a capacity of 1 to 5000 L. Preferably, the bioreactor has a capacity of at least 0.5, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000 or 4000 L. Preferably, the bioreactor has a capacity of at most 5000, 4000, 3000, 2000, 1000, 900, 800, 700, 600, 500, 400, 300, 200, 100, 90, 80, 70, 60, 50, 40, 30, 20, 10, 9, 8, 7, 6, 5, 4, 3, 2 or 1 L. Preferably, the bioreactor used in step b) is not a three-dimensional perfusion bioreactor, in particular a bone marrow mimic.

Preferably, the bioreactor comprises a means of eliminating the used culture medium which allows the cultured cells to be kept. Preferably, this means is a filter, in particular of the spin-filter type (spin or rotary filter), continuous centrifugation, discontinuous centrifugation, or tangential filtration, more preferably the means is of the tangential filtration type. The filter according to the invention makes it possible to eliminate the used culture medium in the form of a permeate, while maintaining the cells cultivated in the bioreactor.

Preferably, the bioreactor used in step b) is thus a tangential flow filtration bioreactor, which may also comprise a continuous or alternating pump. Preferably, the continuous or alternating pump is a low shear pump, which helps preserve the cells.

Preferably, the bioreactor of step b), in particular the tangential flow bioreactor, comprises a filtering member, which may in particular be a filter cassette or a hollow fiber module. Preferably, the bioreactor of step b) is a tangential flow filtration bioreactor equipped with a filtering member comprising a hollow fiber module. Preferably, the cutoff point of the filtering member allows the cells to be retained within the bioreactor. The cutoff or filter pore size is defined as the molar mass of the smallest compound in the filtered medium that is observed to be retained by the filter at 90%. Typically, the cutoff is specified for commercially available filters. Preferably, the cutoff of the filter, in particular of the filtering member, is from 1 kDa to 1.3 μm or 500 kDa. Preferably, the cutoff according to the invention is less than 5 μm, 1.2 μm, 0.22 μm, 0.05 μm, 76 kDa, 70 kDa, 60 kDa, 50 kDa, 40 kDa, 30 kDa, 20 kDa, 15 kDa, 10 kDa, 9 kDa, 8 kDa, 7 kDa, 6 kDa, 5 kDa, 4 kDa, 3 kDa, 2 kDa or 1 kDa. Preferably, the cutoff according to the invention is greater than 1 kDa, 2 kDa, 3 kDa, 4 kDa, 5 kDa, 6 kDa, 7 kDa, 8 kDa, 9 kDa, 10 kDa, 15 kDa, 20 kDa, 30 kDa, 40 kDa, 50 kDa, 60 kDa, 70 kDa, 76 kDa, 0.05 μm, 0.22 μm, 1.2 μm, or 5 μm. Preferably, the cutoff according to the invention is of from 1 kDa and to 50 kDa, more preferably of from 1 kDa to 15 kDa.

Preferably, the bioreactor of step b) comprises a gas exchange means allowing to satisfy the oxygen requirements of the cells and to control the pH by controlling the supply and/or removal of carbon dioxide (CO2). Preferably, the gas exchange means is low shear.

Preferably, at least one, more preferably all, of the following culture conditions are controlled or regulated in step b):
Agitation;
pH; DO;
Temperature;
Volume or level of the bioreactor;
Perfusion rate;
Nutrient input, in particular selected from carbohydrates, amino acids, vitamins and iron;
Growth factor input, in particular selected from EPO, SCF, and Insulin;
Fouling of the bioreactor and clogging of the filtering organs.

Preferably, the culture in step b) is performed for a period of time sufficient to achieve a cell concentration higher than 30 million cells/ml. Preferably this period of time is from 5 days to 25 days, more preferably from 10 days to 20 days.

Preferably, also the culture of step b) is continued until at least 10%, 20%, 30%, 40%, 50%, 60%, 70%, 80% or 90%, more preferably at least 50%, of the cultured cells are enucleated.

Preferably, the culture temperature in step b) is comprised between 33° C. and 40° C., more preferably between 35° C. and 39° C., and even more preferably between 36° C. and 38° C.

Preferably, the culture pH of step b) is comprised between 7 and 8, more preferably between 7.2 and 7.7.

Preferably, the culture DO of step b) is between 1% and 100%, more preferably between 10% and 100%.

The description of the culture medium given for step a) of the method of the invention also applies for the present step b).

Particularly preferably, the culture medium used in step b) comprises a basal culture medium for eukaryotic cells, heparin, in particular in a concentration of from 0.2 to 2 U/ml, plasma, especially in a concentration of from 0.5 to 6% (v/v), transferrin in particular in a concentration of from 100 to 500 μg/ml, Insulin, in particular in a concentration of from 5 to 15 μg/ml, SCF, in particular in a concentration of from 50 to 300 ng/ml, and EPO, in particular in a concentration of from 1 to 5 IU/ml and optionally a glucocorticoid, in particular in a concentration of from 0.5 to 5 μM.

Advantageously, step b) of the method of the invention makes it possible to concentrate the cells to levels unattainable in batch and fed-batch culture, i.e. above 30 million cells/ml and up to 200 million cells/ml. Advantageously also, step b) of the method of the invention allows furthering the differentiation of the cultured cells. Advantageously, at the end of the culture of step b) the rate of enucleated cells exceeds 50%.

### Step c)

The cells obtained in step b) are then purified in step c) to give a population of cultured red blood cells. Step c) of the process according to the invention comprises two operations, a particle sorting operation and a washing operation. The washing operation can be performed either before and/or after the particle sorting operation.

The purpose of step c) is to:
sort the cells to concentrate enucleated cells as much as possible; and
wash the cells to eliminate potentially toxic residues yielded by the method.

Particle sorting increases the rate of enucleated cells, especially by eliminating erythroblasts and any residual myeloid cells. Erythroblasts are cultured cells that have not reached the stage of enucleated cell differentiation, i.e. reticulocytes or red blood cells. Particle sorting also removes cellular wastes, such as cellular debris, DNA and pyrenocytes.

The particulate sorting according to the invention may comprise at least one operation selected from the group consisting of tangential flow filtration, dead-end filtration and elutriation.

Tangential-flow filtration is well known to the person skilled in the art. It is a filtration method that separates the particles of a liquid according to their size. In tangential filtration, the flow of liquid is parallel to the filter, contrary to dead-end filtration in which the flow of liquid is perpendicular to the filter. It is the pressure of the fluid that allows it to pass through the filter. As a result, particles that are small enough pass through the filter, while those that are too large continue on their way through the liquid flow.

Dead-end filtration is well known to the skilled person. Its principle consists in retaining the particles to be eliminated inside a porous network constituting the filter. Filtration relies on 4 mechanisms: (i) particle/wall adhesion forces, (ii) inter-particle adhesion forces, (iii) steric hindrance and (iv) the drag force of the fluid on the particles. Its efficiency depends on the material, the pore size, the type of fiber entanglement and the ratio of the filtration surface to the amount of material to be filtered.

Elutriation is a technique for the separation and particle size analysis of particles of different sizes. Elutriation is based on Stokes' law. A fluid containing cells is sent into a chamber at a known speed where the particles are subjected to a controlled centrifugal force. The particles remain in suspension when the two forces (fluid-driving force and centrifuge force) cancel each other out.

Preferably, the particle sorting operation according to the invention comprises a succession of dead-end filtrations and optionally of elutriation.

The purpose of the washing operation is to lower the quantities of toxic compounds potentially present in the cell culture of step b) below their toxicity threshold.

The washing operation may include one or more centrifugations and/or one or more elutriations.

Centrifugation is well known to the person skilled in the art. It is a process for separating compounds in a mixture based on their density and drag difference by subjecting them to a unidirectional centrifugal force and possibly to an opposing flow.

Preferably, the washing step according to the invention comprises a succession of elutriation operations.

The particle sorting, washing and formulation steps are carried out in a time period of less than 72 hours, more preferably less than 12 hours.

At the end of step c) a population of cultured red blood cells is obtained according to the invention.

### Cultured Red Blood Cells

Preferably, the population of cultured red blood cells obtained by implementing the method according to the invention, or population of cultured red blood cells according to the invention, is produced in 14 to 30 days. More preferably, the population of cultured red blood cells obtained by the implementation of the method according to the invention is produced in about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days or about 30 days.

The cultured red blood cells according to the invention have features similar to those of native reticulocytes. As is well known to the skilled person, reticulocytes are derived from erythroblasts by enucleation. This is illustrated by the following Example. Some cultured red blood cells according to the invention may have features similar to those of native red blood cells.

Preferably, the cultured red blood cells or the population of cultured red blood cells according to the invention, obtained, obtainable or that can be obtained, by the implementation of the method according to the invention, have at least 1, preferably at least 2, 3, 4, 5, 6, 7, 8, 9, 10, 11, 12, 13, 14, or all, of the following features:
A percentage of Hoechst+ lower than 30%, preferably less than 10%, more preferably less than 5%, even more preferably less than 3% and most preferably less than 1%;
Optionally a percentage of CD36+ cells lower than 50%, preferably of from 8% to 22%;
Optionally a percentage of CD71+ cells higher than 50%, preferably of from 79% to 92%;
Optionally a percentage of thiazole orange labeled cells higher than 50%, preferably of from 83% to 95%;
A MCV of from 80 fL to 180 fL, in particular of from 80 to 160 fL, preferably of from 130 to 154 fL;
A MCH higher than 24 μg/cell, preferably higher than 28 μg/cell, more preferably higher than 32 μg/cell and even more preferably higher than 36 μg/cell;
A MCHC higher than 18 g/dl, in particular higher than 19 g/dl, preferably higher than 21 g/dl, more preferably higher than 23 g/dl and even more preferably of from 21 g/dl to 29 g/dl;
A p50 of from 18 mmHg to 28 mmHg, preferably of from 18 mmHg to 22 mmHg, of from 20 mmHg to 27 mmHg, of from 21 mmHg to 26.5 mmHg, of from 22 mmHg to 26 mmHg, of from 23 mmHg to 26 mmHg, or of from 24 mmHg to 26 mmHg;
Optionally a proportion of HbA of from 70% to 100%, preferably of from 74% to 86%;
Optionally a proportion of HbF of 0% to 30%, preferably of from 11.5% to 21%; Optionally a proportion of HbA2 lower than 8%, preferably of from 2% to 5%;
A proportion of HbCO of from 0% to 10%, preferably of from 1.5% to 6.5%;
A MetHb proportion of from 0% to 3%, preferably lower than 0.5%;
A deformability greater than 75% of that of native red blood cells, preferably of from 81.5% to 85.5% of that of native red blood cells;
An ATP content of from 4 to 12 μmol/g Hb, preferably of from 7.5 μmol/g Hb to 10.5 μmol/g Hb.

Preferably, the above features comprise:
a percentage of CD36+ cells lower than 50%, preferably of from 8% to 22%;
a percentage of CD71+ cells higher than 50%, preferably of from 79% to 92%;
a percentage of cells labeled with thiazole orange higher than 50%, preferably of from 83% to 95%.

As the person skilled in the art will well understand, when the above features, or second list of features, are added to the foregoing features, or first list of features, the cultured red blood cells or population of cultured red blood cells according to the invention, obtained, obtainable or that can be obtained, by the implementation of the method according to the invention, have at least 1, preferably at least 2, 3, 4, 5, 6, 7, 8, 9, 10, 11, or all, of the features.

Also preferably, the above features comprise:
a proportion of HbA of from 70% to 100%, preferably of from 74% to 86%;
a proportion of HbF of from 0% to 30%, preferably of from 11.5% to 21%;
a proportion of HbA2 lower than 8%, preferably of from 2% to 5%;

As the person skilled in the art will appreciate, when the above features, or third list of features, are added to the preceding features, the first list of features and, if applicable, the second list of features, the cultured red blood cells or the population of cultured red blood cells according to the invention, obtained, obtainable or that can be obtained by implementing the method according to the invention, have at least 1, preferably at least 2, 3, 4, 5, 6, 7, 8, 9, 10, 11, 12 and where applicable 13, 14, or all, of the features.

The percentages (in number) of Hoechst+, CD36+, CD71+, and thiazole orange (TO+) labeled cells can be determined by flow cytometry, according to techniques well known to the person skilled in the art, in particular with the help of a FACSCalibur apparatus (BD Biosciences) with the Cell Quest software.

The MCV, MCH and MCHC measurements are standard hematology measurements that can be determined by commercial automats, such as the XN 9100 (Sysmex).

The p50 or partial pressure of oxygen at which the hemoglobin oxygen saturation is 50% can be determined by techniques well known to the person skilled in the art and in particular by using a Hemox Analyzer (TCS).

The percentages (m/m) of HbA, HbF and HbA2 refer respectively to the amount (in mass) of hemoglobin (Hb) considered in relation to the total amount of hemoglobin (in mass). These percentages can be determined by high performance liquid chromatography (HPLC) on a cation-exchange column, particularly as described by Ou & Rognerud (1993) “Rapid analysis of hemoglobin variants by cation-exchange HPLC” Clinical Chemistry 39: 820-824 (incorporated herein by reference).

The percentages (m/m) of HbCO, i.e. hemoglobin bound to carbon monoxide, and MetHb, i.e. methemoglobin, can be determined by techniques well known to the person skilled in the art, in particular with the aid of a blood gas analyzer, such as Rapidlab (Siemens)

The percentage of deformability can be determined using the LORRCA equipment, in particular as described in the article Giarratana et al. (2011) “Proof of principle for transfusion of in vitro-generated red blood cells”, Blood 118:5071-5079 (incorporated herein by reference), page 5072, paragraph “Deformability measurements”. Deformability is expressed as a percentage of the deformability of cultured red blood cells relative to the deformability of native red blood cells, i.e., peripheral red blood cells from a donor of the same species as the cultured red blood cells, in particular a human donor, especially an adult donor, for human cultured red blood cells.

The ATP content can be determined colorimetrically or fluorimetrically, in particular by measuring the product of the reaction of glycerol with ATP, the amount of which can be measured colorimetrically or fluorimetrically and is proportional to the amount of ATP, for example using a MAK190 kit (Sigma). The ATP content is expressed in μmol and divided by the total amount (in g) of hemoglobin.

In some embodiments that may be combined with any of the foregoing embodiments, the cultured red blood cell population according to the invention is a human cell population.

In some embodiments which may be combined with any of the foregoing embodiments, the cultured red blood cell population according to the invention has one or more blood groups selected from A+, A−, B+, B−, AB+, AB−, O+ and

In some embodiments that may be combined with any of the foregoing embodiments, the cultured red blood cells according to the invention have a rare or universal blood type.

According to an embodiment of the invention, the cultured red blood cell population according to the invention is formulated in a red blood cell preservation solution. Any formulation known in the state of the art for preserving a red blood cell population may be used.

### Therapeutic Application

Any pharmaceutically acceptable excipient or vehicle known in the art that is suitable for use with red blood cells may be used. As an example, the pharmaceutically acceptable excipient or vehicle is a pH balanced saline solution.

The pharmaceutical compositions according to the invention may also comprise one or more exogenous proteins of interest useful in the prevention, treatment or diagnosis of one or more diseases or disorders, in particular related to a deficiency or a lack of red blood cells or of functional hemoglobin.

Any formulation known in the art for administering to an individual a pharmaceutical composition containing a population of red blood cells may be used. Preferably, the pharmaceutical composition according to the invention is formulated as a blood transfusion bag.

The individual according to the invention is an animal, preferably a mammal, more preferably a human.

The invention will be further explained with the following non-limiting Example.

### Example

The cultured red blood cells obtained by the implementation of the method according to the invention were compared to native red blood cells, more particularly to placental blood reticulocytes.

Briefly, the cells cultured according to the invention are total nucleated cells collected by cytapheresis from voluntary donors previously mobilized with G-CSF.

Step a) of the process according to the invention is conducted over 7 days in fed-batch at a temperature of 37° C., under a 5% CO2 atmosphere and in a culture medium adapted from that described by Giarratana et al. (2011) “Proof of principle for transfusion of in vitro-generated red blood cells”, Blood 118:5071-5079 for the first step of the expansion procedure described in the article (page 5072). Halfway through step a) fresh culture medium is added to the culture in order to dilute the culture by half (the same volume of culture medium is added as the volume initially present).

Step b) of the process according to the invention is carried out over 15 days in a perfusion bioreactor at a temperature of 37° C., under an atmosphere of 5% CO2, with a culture medium similar to that of step a) except that IL-3 and glucocorticoid are absent. Occasional additions of SCF and EPO are also made as well as a continuous supply in iron.

Step c) of the process according to the invention is carried out by performing a particle sorting by a succession of dead-end filtrations, followed by elutriation cell washing.

The features of the resulting cultured red blood cell population were determined and are summarized in Table 1 below.

TABLE 1

| | | | Native | |
|---|---|---|---|---|
| Criterion | Mean | σ | reticulocyte* | |
| CD36 (%) | 14.9 | 6.8 | 22 ± 15 | (n = 3) |
| CD71 (%) | 85.5 | 6.5 | 90 ± 2 | (n = 3) |
| Thiazole orange (%) | 89 | 6 | 89 ± 7 | (n = 6) |
| MCV (Mean Corpuscular Volume) | 141.9 | 11.5 | 103-130 | |
| MCH (pg Hb/cell) | 34.3 | 4.1 | 24-31 | |
| MCHC (mean corpuscular hb | 24.2 | 2.4 | 26-31 | |
| concentration - g/dl) | | | | |
| p50 (mmHg)Hb | 20.8 | 1.2 | 20 | |
| % HbA | 78.4 | 4.3 | 22 ± 8.5 | (n = 7) |
| % HbF | 14.9 | 3.4 | 78 ± 8.5 | (n = 7) |
| % HbA2 | 3.4 | 1.2 | 0.7 | (n = 1) |
| % HbCO | 4.2 | 2.3 | 10 | (n = 1) |
| % MetHb | 0.3 | 0.1 | 2.7 | (n = 1) |
| Deformability (%/control | 83 | 2.3 | 86-88 | |
| native red blood cells) | | | | |
| ATP | 8.5 | 1.9 | 8 | (literature) |

*Reticulocytes from placental blood

The means and standard deviations obtained during the cultures are compared to the expected values for native reticulocytes. These indicators verify that:
The expressed phenotypes correspond well to those of reticulocytes (CD71 and CD36);
The presence of residual nucleic acids is consistent with reticulocyte status (TO);
Cells have an amount of hemoglobin sufficient and functional for oxygen transport (MCH, MCHC, HbF, HbA, HbA2 and P50);
The HbCO and MetHb levels are within the physiologically accepted norms of healthy individuals;
The cells have a sufficient reserve of energy to ensure maintenance (ATP);

The cells are deformable and of reasonable size to ensure good blood circulation (Deformability, MCV); The only significant difference with the native reticulocytes studied is the reversed content of fetal and adult Hb. This difference is justified by the fact that cultured red blood cells are now produced from adult stem cells (and therefore contain mostly adult hemoglobin), while control reticulocytes are derived from placental blood (and therefore contain mostly fetal hemoglobin).

All these criteria have been measured systematically for several implementations of the method according to the invention, which allows a study of the stability of the method (see column CV: coefficient of variation). These results show that the synthetic red blood cells have features very close to native reticulocytes (except for the repartition between HbA and HbF which is reversed). In addition, the method is stable compared to the variability of measurements made on native reticulocytes.

The method according to the invention produces cultured red blood cells with features close to native reticulocytes. In addition, the low variability of the measurements performed indicates that the risk of deviating from the features of native reticulocytes is low

Furthermore, the method according to the invention allows to significantly improve the concentration of cultured red blood cells in the resulting population with in the range of 50 to 130 million cells per ml compared to less than 5 million cells per ml with the prior art method described by Giarratana et al. (2011) “Proof of principle for transfusion of in vitro-generated red blood cells”, Blood 118:5071-5079.

It is further estimated that while it would take on the order of 9000 175 cm² flasks such as those used in the article Giarratana et al. (2011) “Proof of principle for transfusion of in vitro-generated red blood cells”, Blood 118:5071-5079 to produce one unit of transfusion of cultured red blood cells, which underlines the difficulty of industrial implementation of the method of the prior art, the implementation of the method according to the invention would allow to obtain the same quantity of cultured red blood cells in one time with the help of a perfusion bioreactor of a few tens of liters capacity.

1. A method of producing cultured red blood cells from stem cells or cells of an immortalized cell line of the erythroid lineage, comprising the following steps:
a) culturing the cells in at least one batch or fed-batch bioreactor;
b) culturing the cells obtained in step a) via a perfusion bioreactor; and
c) washing and particle sorting of the cells obtained in step b), thereby producing a population of cultured red blood cells.

2. The method according to claim 1, wherein the cells are embryonic stem cells (ESCs), pluripotent stem cells (iPSCs), or hematopoietic stem cells and/or progenitors (HSCs/HPs).

3. The method according to claim 1, wherein said cells are cells of an immortalized cell line of the erythroid lineage.

4. The method according to claim 1, wherein the cells are from umbilical cord/placental blood, peripheral blood, bone marrow, or apheresis collection.

5. The method according to claim 1, wherein step a) is carried out for a period of time sufficient to obtain a cell concentration higher than 0.1 million cells/ml.

6. The method according to claim 1, wherein step b) is carried out for a period of time sufficient to obtain a cell concentration at a level higher than 30 million cells/ml.

7. The method according to claim 1, wherein particle sorting comprises a succession of dead-end filtrations and optionally elutriation.

8. The method according to claim 1, wherein the washing comprises one or more centrifugations and/or one or more elutriations.

9. A population of cultured red blood cells obtainable by carrying out the method according to claim 1.

10. A population of cultured red blood cells having at least 6 of the following features:
a percentage of Hoechst+ lower than 30%;
a MCV of from 80 fL to 180 fL;
a MCH higher than 24 μg/cell;
a MCHC higher than 18 g/dl;
a p50 of from 18 to 28 mmHg;
a proportion of HbCO of from 0% to 10%;
a MetHb proportion of from 0% to 3%;
a deformability higher than 75% of that of native red blood cells; and/or
an ATP content of from 4 to 12 μmol/g Hb.

11. The cultured red blood cell population of claim 10, wherein the features further comprise:
a percentage of CD36+ cells lower than 50%;
a percentage of CD71+ cells higher than 50%; and/or
a percentage of cells labelled with thiazole orange higher than 50%.

12. The cultured red blood cell population of claim 10, wherein the features further comprise:
a proportion of HbA of from 70% to 100%;
a proportion of HbF of from 0% to 30%; and/or
a proportion of HbA2 lower than 8%.

13. A pharmaceutical composition comprising a population of cultured red blood cells according to claim 9 as active substance, optionally in association with at least one pharmaceutically acceptable carrier or excipient.

14. The method according to claim 1, wherein said cells erythroid progenitors or early erythroid precursors.

15. The method according to claim 5, the period of time to obtain a cell concentration higher than 0.1 million cells/ml is from 1 to 15 days.

16. The method according to claim 5, the period of time to obtain a cell concentration higher than 0.1 million cells/ml is from 3 to 10 days.

17. The method according to claim 6, the period of time to obtain a cell concentration at a level higher than 30 million cells/ml is from 5 days to 25 days.

18. The method according to claim 6, the period of time to obtain a cell concentration at a level higher than 30 million cells/ml is from 10 days to 20 days.

19. The population of cultured red blood cells according to claim 10, comprising all of the following features:
a percentage of Hoechst+ lower than 30%;
a MCV of from 80 fL to 180 fL;
a MCH higher than 24 μg/cell;
a MCHC higher than 18 g/dl;
a p50 of from 18 to 28 mmHg;
a proportion of HbCO of from 0% to 10%;
a MetHb proportion of from 0% to 3%;
a deformability higher than 75% of that of native red blood cells; and
an ATP content of from 4 to 12 μmol/g Hb.
